(19) United States
(12) Patent Application Publication
(10) Pub. No.: US 2024/0019436 A1
(43) Pub. Date: Jan. 18, 2024

(54) BIOMARKERS FOR MYCOBACTERIUM AVIUM PARATUBERCULOSIS

(71) Applicant: ABERYSTWYTH UNIVERSITY, Ceredigion Aberystwyth (GB)

(72) Inventors: Luis Alejando Jose Mur, Ceredigion (GB); Emma Nicole Taylor, Bury Greater Manachester (GB); David John Rooke, Horsham West Sussex (GB); Adriaan Peter Koets, Marknesse (NL)

(21) Appl. No.: 18/038,901

(22) PCT Filed: Nov. 24, 2021
(86) PCT No.: PCT/GB2021/053054

§ 371 Date: May 25, 2023

(30) Foreign Application Priority Data
Nov. 25, 2020 (GB) 2018541.9

(51) Int. Cl. G01N 33/569

(52) U.S. Cl. CPC G01N 33/5695; G01N 2800/065; G01N 2570/00

(57) ABSTRACT

The present invention relates to a method for determining the presence of Mycobacterium avium paratuberculosis in a subject. The method comprises: (i) determining the level of one or more metabolites in a sample from the subject; and (ii) comparing the level of said one or more metabolites with the level of said one or more metabolites in a control sample to determine whether Mycobacterium aviumparatuberculosis is present in the subject. The metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-non-adecenoic acid; creatinine; 5-hydroxyeicosapentaenoic acid, 17-hydroxy docosahexaenoic acid, palmitoleic acid; R-3-hydroxy-octade-canoic acid; 8, 11, 14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14, 17-eicosatetraenoic acid; palmitic acid; pyruvic acid; and p-cresol.

### FIELD OF THE INVENTION

The present invention relates to biomarkers for detecting Mycobacterium avium paratuberculosis, the causative agent of paratuberculosis. In particular, the present invention relates to a method for determining the presence of Mycobacterium avium paratuberculosis in a subject. The present invention further relates to a device for detecting Mycobacterium avium paratuberculosis in a subject and a kit for detecting Mycobacterium avium paratuberculosis in a subject.

### BACKGROUND TO THE INVENTION

Paratuberculosis, commonly known as Johne's disease, is a chronic intestinal infection in ruminants, caused by Mycobacterium avium subspecies paratuberculosis (MAP). MAP is commonly transmitted via infected faeces or colostrum, but other modes of transmission include in-utero, semen and contaminated environments, like manure, soil or stream water. The progression of MAP infections can be subdivided into the following phases: incubation period; subclinical stage; and clinical stages.

Infected cattle begin shedding MAP at the subclinical stage, via faeces and milk. However, visible symptoms, including weight loss, diarrhoea, elevated thirst and reduced milk yields are absent until 2 to 10 years post-infection during the clinical stages. Therefore, MAP can be spread when no clinical symptoms are apparent. The financial impacts of MAP infections are significant through milk yield loss and voluntary culling (Barratt et al., 2018). Improved disease detection, and subsequent control, would benefit cattle health, welfare and reduce the economic impact on farmers.

Detecting the presence of MAP infections is challenging as only 10-15% of MAP infected cattle display clinical signs and the performance of diagnostic tests is dependent on the stage of infection. The current antibody-based tests are based on enzyme-linked immunosorbent assay (ELISA). However, the sensitivity of ELISA tests against a series of serum protein antigens is very variable, ranging from 28-94%, and specificity from 41-100%. Faecal culture, milk ELISA antibody and interferon-γ tests exhibit similar problems. Consequently, repeat testing is required to accurately assess the MAP status of herds. A UK-based survey using ELISA tests estimated herd prevalence at 34.7% (credible interval 27.6-42.5%) (DEFRA et al., 2009) but this is most likely an underestimate.

Johne's disease is a significant problem in cattle herds that, as a chronic disease, has not the prominence accorded to diseases such as bovine tuberculosis. However, it represents a significant source of financial loss to cattle farmers and current approaches for identifying MAP infection are ineffective, particularly at the subclinical stage.

Crohn's disease is an inflammatory bowel disease in humans which causes inflammation of the digestive tract resulting in abdominal pain, severe diarrhoea, fatigue and weight loss. The exact cause of Crohn's disease is unknown. However, studies have suggested a link between Mycobacterium avium paratuberculosis and Crohn's disease. For example, a study detected MAP in 28 individuals with Crohn's disease (Naser et al., 2004). Crohn's disease can often go undiagnosed for long periods of time and it would be advantageous to diagnose the presence of this disease at an early stage so that the patient can be managed and treated accordingly.

It is an object of the present invention to obviate or mitigate one or more of the abovementioned problems.

### SUMMARY OF THE INVENTION

The present invention relates to a method for determining the presence of Mycobacterium avium paratuberculosis in a subject and is based, in part, on studies by the inventors in which they have shown that certain metabolites are present at significantly different levels in naturally MAP infected heifers as compared to control heifers and are therefore suitable as biomarkers for paratuberculosis and/or Crohn's disease.

In a first aspect of the present invention there is provided a method for determining the presence of Mycobacterium avium paratuberculosis in a subject, the method comprising the steps of:
(i) determining the level of one or more metabolites in a sample from the subject;
(ii) comparing the level of said one or more metabolites with the level of said one or more metabolites in a control sample to determine whether Mycobacterium avium paratuberculosis is present in the subject;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

As discussed in more detail below, the present inventors have found that the level of the abovementioned metabolites is significantly altered in subjects in which Mycobacterium avium paratuberculosis is present. As shown below, the inventors have found that these metabolites have individual diagnostic accuracies (area under the curve; AUC) of over 70%. The inventors have therefore found that by comparing the level of the abovementioned metabolites in a sample from a subject with the level in a control sample, the presence of Mycobacterium avium paratuberculosis in a subject can be determined with high sensitivity and specificity. These metabolite ‘biomarkers’ are therefore useful for determining whether a subject has paratuberculosis, particularly subclinical paratuberculosis. By quickly and accurately identifying animals affected by paratuberculosis at an early stage of infection, and before clinical symptoms become apparent, spread of the disease can be reduced and/or prevented.

In embodiments, the one or more metabolites are selected from 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; and phytanic acid. The inventors have found that these metabolites have individual diagnostic accuracies (area under the curve; AUC) of over 75%.

In embodiments, the one or more metabolites are selected from 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 17-hydroxy docosahexaenoic acid; palmitoleic acid; R-3-hydroxy-octadecanoic acid; 8,11,14-eicosatrienoic acid; nonadecanoic acid; and phytanic acid. The inventors have found that these metabolites have individual diagnostic accuracies (area under the curve; AUC) of over 80%.

In embodiments, the one or more metabolites are selected from 2-hydroxyglutaric acid; leukotriene B4; itaconic acid; bicyclo-prostaglandin E2; N6-acetyl-L-lysine;

guanidinobutanoic acid; creatine; docosahexaenoic acid; and cis-10-nonadecenoic acid. The inventors have found that these metabolites have individual diagnostic accuracies (area under the curve; AUC) of over 85%.

In preferred embodiments, the one or more metabolites are selected from 2-hydroxyglutaric acid; leukotriene B4; itaconic acid; bicyclo-prostaglandin E2; N6-acetyl-L-lysine; and guanidinobutanoic acid. The inventors have found that these metabolites have individual diagnostic accuracies (area under the curve; AUC) of over 90%.

In even more preferred embodiments, the one or more metabolites are selected from 2-hydroxyglutaric acid; leukotriene B4; itaconic acid; bicyclo-prostaglandin E2; and N6-acetyl-L-lysine. The inventors have found that these metabolites have individual diagnostic accuracies (area under the curve; AUC) of over 95%. The levels of these metabolites have been shown by the inventors to be consistently and significantly different between naturally MAP infected heifers and control heifers. These metabolite biomarkers can therefore be used to determine the presence of Mycobacterium avium paratuberculosis in a subject with extremely high sensitivity and specificity.

The method of the present invention comprises determining the level of one or more metabolites in a sample from the subject. Preferably, the method of the present invention comprises determining the level of two, three, four, five or six (or more) metabolites in a sample from the subject. By determining the level of multiple metabolites in the sample and comparing these levels with the levels in a control sample, the sensitivity and specificity of the determination (of whether a subject has Mycobacterium avium paratuberculosis) may be improved. The level of one or more metabolites in the sample will be determined. Together these metabolite levels may be integrated to form a “fingerprint” for the sample, thereby increasing both the specificity and sensitivity of the method.

For example, in preferred embodiments of the invention, the method comprises determining the level of one or more of the following metabolites: 2-hydroxyglutaric acid; leukotriene B4; itaconic acid; bicyclo-prostaglandin E2; and N6-acetyl-L-lysine. Preferably, the method comprises determining the levels of two, three, four or five of these metabolites.

In embodiments in which the method involves determining the level of six metabolites in a sample from the subject, the metabolites may consist of 2-hydroxyglutaric acid; leukotriene B4; itaconic acid; bicyclo-prostaglandin E2; N6-acetyl-L-lysine; and guanidinobutanoic acid.

In embodiments in which the method involves determining the level of five metabolites in a sample from the subject, the metabolites may consist of 2-hydroxyglutaric acid; leukotriene B4; itaconic acid; bicyclo-prostaglandin E2; and N6-acetyl-L-lysine.

In preferred embodiments, the method of the present invention comprises the steps of:
(i) determining the level of five metabolites in a sample from the subject;
(ii) comparing the level of said five metabolites with the level of said five metabolites in a control sample to determine whether Mycobacterium avium paratuberculosis is present in the subject;

wherein the metabolites consist of 2-hydroxyglutaric acid; leukotriene B4; itaconic acid; bicyclo-prostaglandin E2; and N6-acetyl-L-lysine.

The present inventors have found that using these five metabolites, in combination, offers a “signature” or “diagnostic fingerprint” which allows the highly accurate identification of Mycobacterium avium paratuberculosis.

The method of the present invention involves determining the level of one or more metabolites in a sample. The skilled person will appreciate that there are a number of ways in which a metabolite level can be determined.

For example, the metabolite level may be determined using mass spectrometry, for example high resolution mass spectrometry (HR-MS), gas chromatography time-of-flight mass spectrometry (GC-MS), flow infusion electrospray high resolution mass spectrometry (FIE-HRMS) or liquid chromatography-electrospray mass spectrometry (LC-MS).

GC-MS involves linking a gas chromatograph with a mass spectrometer. The gas chromatograph utilizes a capillary column where the chemical properties between the sampled chemicals a mixture and their relative affinity for the stationary phase of the column will result in their separation along the column. This provides “retention time”, information. The chemicals then enter the mass spectrometer which will show mass-to-charge ratios.

LC-MS links liquid chromatography (LC or High Performance LC [HPLC]) with a mass spectrometer. The LC part physically separates chemicals between a liquid mixture of two immiscible phases, i.e., stationary and mobile. The chemicals then enter the mass spectrometer which will show mass-to-charge ratios.

In flow infusion, samples are injected directly into a solvent (usually methanol-water) line leading to a mass spectrometer.

Alternatively, the metabolite level may be determined using NMR, enzymatic assays (e.g. enzymatic reaction followed by colorimetric detection) or immunoassays (i.e. antibody binding based assays).

The most well-established immunoassay is the enzyme-linked immunosorbent assay (ELISA). The most used ELISA technique is the sandwich ELISA which measures the antigen using a capture (often associated within the well of a 96 well plate to allow high-throughput screening) and a detection antibody. Monoclonal or polyclonal antibodies can be used in sandwich or competitive ELISA systems. Other immunoassay systems include lateral flow or flow through systems. The use of such immunoassays for detecting the metabolite level would allow the method to be used as a Point of Care (POC) test, for example a POC device, allowing the presence of MAP to be determined in locations away from a laboratory, for example at a farm or remote location.

The step of determining the metabolite level (and comparing the metabolite level with the metabolite level in a control sample) may be performed using a POC test device, for example the device described below. For example, the step of determining the metabolite level (and comparing the metabolite level with the metabolite level in a control sample) may be performed using a flow through device or a lateral flow device. In embodiments, the step of determining the metabolite level (and comparing the metabolite level with the metabolite level in a control sample) may be performed using a lateral flow device, for example the device described below.

The term “metabolite level” as used herein is used to mean the amount of metabolite present in the samples. As will be appreciated by the skilled person, this would be standardised with a set volume of sample used in the assay and the presence of internal controls, for example the detection of serum albumin levels (so that metabolite levels are determined relative to this established standard).

Step (ii) of the method of the present invention involves comparing the level of said one or more metabolites with the level of said one or more metabolites in a control sample to determine whether Mycobacterium avium paratuberculosis is present in the subject.

As will be appreciated by the skilled person, the origin of the control sample will depend upon the particular subject being tested. However, the control sample may be obtained from an age-matched subject and/or a subject of the same sex. Furthermore, since the comparison of metabolite levels may be used to determine whether Mycobacterium avium paratuberculosis is present in a subject, the control sample may be derived, for example, from a subject in which Mycobacterium avium paratuberculosis is not present. Since the comparison of metabolite levels may subsequently be used to determine whether a subject has paratuberculosis (including subclinical paratuberculosis) or Crohn's disease, the control sample by be derived from a subject who does not have paratuberculosis or Crohn's disease.

In the method of the present invention the metabolite level in a sample from a subject is compared with the metabolite level in a sample from a control. As will be appreciated by the skilled person, in the comparison step, the level of a metabolite in the sample is compared with a corresponding metabolite level in the control (e.g. in embodiments in which the metabolite leukotriene B4 is utilised, the level of leukotriene B4 in the sample is compared with the level of leukotriene B4 in the control).

In embodiments of the invention, a metabolite level in the sample from the subject which is higher or lower than the metabolite level in the control sample indicates that Mycobacterium avium paratuberculosis is present in the subject.

In embodiments of the invention in which the one or more metabolites are selected from: 16-methylheptadecanoic acid; 17-hydroxy docosahexaenoic acid; 2-hydroxyglutaric acid; R-3-hydroxy-octadecanoic acid; guanidinobutanoic acid; 8,11,14,17-eicosatetraenoic acid; 8,11,14-eicosatrienoic acid; 9,10-dihydroxy-12-octadecenoic acid; nonadecanoic acid; arachidonic acid; bicyclo-prostaglandin E2; creatinine; creatine; docosahexaenoic acid; eicosapentaenoic acid; itaconic acid; N6-acetyl-L-lysine; leukotriene B4; palmitic acid; palmitoleic acid; p-cresol; phytanic acid; pyruvic acid; stearic acid; and cis-10-nonadecenoic acid, a metabolite level in the sample from the subject which is higher than the metabolite level in the control sample indicates that Mycobacterium avium paratuberculosis is present in the subject.

In embodiments of the invention in which the one or more metabolites is 5-hydroxyeicosapentaenoic acid, a metabolite level in the sample from the subject which is lower than the metabolite level in the control sample indicates that Mycobacterium avium paratuberculosis is present in the subject.

In preferred embodiments of the invention in which the one or more metabolites comprise 2-hydroxyglutaric acid; leukotriene B4; itaconic acid; bicyclo-prostaglandin E2; N6-acetyl-L-lysine; and/or guanidinobutanoic acid, a metabolite level in the sample which is higher than the metabolite level in the control indicates that Mycobacterium avium paratuberculosis is present in the subject.

As will be appreciated by the skilled person, when referring to a higher or lower metabolite level in a sample compared to a control, we mean a metabolite level which is significantly higher (at least two-fold higher) or significantly lower (at least 0.5-fold lower) than the metabolite level in a control.

In embodiments in which the metabolite level in the sample from the subject is higher than the metabolite level in the control sample, preferably the metabolite level in the sample is at least two-fold higher than in the control. More preferably, the metabolite level in the sample is at least three-fold, four-fold or five-fold higher than in the control.

More preferably, the metabolite level in the sample is at least six-fold, seven-fold, eight-fold, nine-fold or ten-fold higher than in the control.

In embodiments in which the metabolite level in the sample from the subject is lower than the metabolite level in the control sample, preferably the metabolite level in the sample is at least 0.5-fold lower than in the control.

The present invention provides a method for determining the presence of Mycobacterium avium paratuberculosis in a subject. As discussed above, Mycobacterium avium paratuberculosis causes paratuberculosis, a chronic disease which primarily affects ruminants, although cases have also been described in a variety of other animals including, for example, rabbits, foxes, and birds.

In embodiments of the present invention, the subject is an animal, for example, a rabbit, fox, bird or ruminant. In preferred embodiments, the animal is a ruminant. A ruminant (or ruminant like) animal is a mammal and for the purposes of the present invention includes cattle, sheep, goats, giraffes, yaks, deer, antelope, camels, buffalo, elk, bison, moose, alpacas, llamas, gazelles, pronghorns, okapis and chevrotains.

In embodiments of the present invention, the subject is selected from cattle, sheep or goats. Preferably, the subject is domestic cattle, which includes beef cattle and dairy cows for example.

The presence of Mycobacterium avium paratuberculosis has also been detected in subjects with Crohn's disease.

In embodiments of the present invention, the subject may be a human.

The method of the present invention may further comprise providing a sample from a subject. In such embodiments the method of the present invention comprises the steps of:
(i) providing a sample from a subject;
(ii) determining the level of one or more metabolites in the sample; and
(iii) comparing the level of said one or more metabolites with the level of said one or more metabolites in a control sample to determine whether Mycobacterium avium paratuberculosis is present in the subject;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The present invention comprises determining the level of one or more metabolites in a sample from the subject. As will be appreciated by the skilled person, the sample may comprise a biological sample from the subject. The biological sample may have been obtained from a bodily fluid of the subject (e.g. a ruminant). The biological sample, may include, for example, blood and blood components (e.g. serum), mucus, saliva, urine, vomit, faeces, sweat, semen, vaginal secretion, tears, pus or milk. Preferably the biological sample is a blood sample. As will be appreciated by the skilled person, the method of the present invention is therefore an in vitro method for determining the presence of Mycobacterium avium paratuberculosis in a subject, the method being carried out on a sample provided from a subject.

In a further aspect of the invention there is provided a method for determining the presence of Mycobacterium avium paratuberculosis in a subject, the method comprising the steps of:
(i) obtaining a sample from the subject;
(ii) determining the level of one or more metabolites in the sample;
(iii) comparing the level of said one or more metabolites with the level of said one or more metabolites in a control sample to determine whether Mycobacterium avium paratuberculosis is present in the subject;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The method of obtaining the sample from the subject will depend on the sample type and subject. In embodiments in which the sample is a blood sample, the step of obtaining the sample may comprise using a needle and syringe or a double-pointed needle and vacutainer tube.

There is also provided a method for determining the presence of paratuberculosis in a subject, determining progression of paratuberculosis or assessing response to therapy of a subject with paratuberculosis. The method comprises the steps of:
(i) determining the level of one or more metabolites in a sample from the subject;
(ii) comparing the level of said one or more metabolites with the level of said one or more metabolites in a control sample to determine whether the subject has paratuberculosis, has paratuberculosis which is progressing, or is responding to therapy for paratuberculosis;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

There is also provided a method for determining the presence of paratuberculosis in a subject, determining progression of paratuberculosis or assessing response to therapy of a subject with paratuberculosis. The method comprises the steps of:
(i) obtaining a sample from the subject;
(ii) determining the level of one or more metabolites in a sample from the subject;
(iii) comparing the level of said one or more metabolites with the level of said one or more metabolites in a control sample to determine whether the subject has paratuberculosis, has paratuberculosis which is progressing, or is responding to therapy for paratuberculosis;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

There is also provided a method for determining the presence of Crohn's disease in a subject, determining progression of Crohn's disease or assessing response to therapy of a subject with Crohn's disease. The method comprises the steps of:
(i) determining the level of one or more metabolites in a sample from the subject;
(ii) comparing the level of said one or more metabolites with the level of said one or more metabolites in a control sample to determine whether the subject has Crohn's disease, has Crohn's disease which is progressing, or is responding to therapy for Crohn's disease;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

There is also provided a method for determining the presence of Crohn's disease in a subject, determining progression of Crohn's disease or assessing response to therapy of a subject with Crohn's disease. The method comprises the steps of:
(i) obtaining a sample from the subject;
(ii) determining the level of one or more metabolites in a sample from the subject;
(iii) comparing the level of said one or more metabolites with the level of said one or more metabolites in a control sample to determine whether the subject has Crohn's disease, has Crohn's disease which is progressing, or is responding to therapy for Crohn's disease;

wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

As discussed above, the presence of Mycobacterium avium paratuberculosis has been detected in subjects with Crohn's disease and is considered a potential cause of Crohn's disease. Therefore, by detecting the presence of Mycobacterium avium paratuberculosis in a subject, it may be possible to determine the presence of Crohn's disease in said subject.

In embodiments where the method is being used to determine whether paratuberculosis or Crohn's disease is progressing, the method may comprise monitoring over time to determine whether paratuberculosis or Crohn's disease has progressed. In such an embodiment, an initial metabolite level (as described above) may be compared with a metabolite level in a sample obtained later in time. The metabolite level(s) obtained may change over time, to be further removed (either higher or lower) from a control metabolite level. Alternatively, in examples where multiple metabolites are utilised, for example five metabolites, in an initial analysis only three of the five metabolites may be indicative of paratuberculosis or Crohn's disease. In the later analysis an increased number of metabolites may be indicative of paratuberculosis or Crohn's disease, which may indicate that paratuberculosis or Crohn's disease is progressing.

In embodiments where the method is being used to determine whether a subject is responding to therapy, the method may comprise monitoring over time to determine whether a subject is responding to therapy. In such an embodiment, an initial metabolite analysis (which may be before therapy has commenced) may be compared with one or more metabolite analyses undertaken on samples obtained later in time (for example after therapy has commenced). The metabolite levels may change over time, to be further removed (either higher or lower) from a control metabolite level. Alternatively, in examples where multiple metabolites are utilised, for example five metabolites, in an initial analysis, five out of five metabolite levels may be indicative of paratuberculosis or Crohn's disease. In a later analysis, fewer metabolites may be indicative of paratuberculosis or Crohn's disease, which may indicate that the subject is responding to therapy. Alternatively, in a later analysis, the number of metabolites indicative of paratuberculosis or Crohn's disease may remain the same or increase, which may indicate that a subject is not responding to therapy.

The method of the present invention may also be used to determine whether a drug is effective at treating paratuberculosis and/or Crohn's disease, in a similar manner to that described above in relation to determining whether a subject is responding to therapy. The use of “effective” is used to indicate that a treatment reduces or alleviates signs or symptoms of paratuberculosis and/or Crohn's disease, improves the clinical course of the disease, decreases the number or severity of exacerbations or reduces any other objective or subjective indicia of the disease. The method of the present invention can be used to determine whether drugs used to treat paratuberculosis and/or Crohn's disease, in addition to other drugs developed to treat paratuberculosis and/or Crohn's disease are effective.

Since a diagnosis of a disease is often not based on the results of a single test alone, the method of the present invention may be used to determine whether a subject is more likely than not to have paratuberculosis or Crohn's disease based on comparison of one or more metabolite levels with a control metabolite level. Thus, for example, a subject with a putative diagnosis of paratuberculosis or Crohn's disease may be diagnosed as being “more likely” or “less likely” to have paratuberculosis or Crohn's disease in light of the information provided by the method of the present invention. The present invention may therefore be used to assist a clinician/veterinarian with the diagnosis of paratuberculosis or Crohn's disease.

The method of the present invention may, in certain embodiments, comprise detecting other signs or symptoms of paratuberculosis or Crohn's disease, conducting clinical tests of paratuberculosis or Crohn's disease and/or measuring other paratuberculosis or Crohn's disease markers.

As will be appreciated by the skilled person, the above description is not limited to making an initial identification (or diagnosis) of paratuberculosis or Crohn's disease in a subject but is also applicable to confirming a provisional diagnosis of paratuberculosis or Crohn's disease or “ruling out” such a diagnosis.

The present invention also provides an immunological capture device for detecting Mycobacterium avium paratuberculosis in a subject, the device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The present invention also provides a immunological capture device for detecting paratuberculosis in a subject, the device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The present invention also provides a immunological capture device for detecting Crohn's disease in a subject, the device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The term “immunological capture device” as described herein is used to describe an immunoassay device which can be used to measure the presence of a metabolite in a sample through the use of an antibody. The antibody would be specific to the metabolite of interest, such that the antibody could “capture” the metabolite, through binding, if the metabolite is present. The antibodies may therefore be described as “capture antibodies”.

Preferably, the device comprises antibodies to one or more of the following metabolites: 2-hydroxyglutaric acid; leukotriene B4; itaconic acid; bicyclo-prostaglandin E2; N6-acetyl-L-lysine. Preferably, the device comprises antibodies to two, three, four or five of the following metabolites: 2-hydroxyglutaric acid; leukotriene B4; itaconic acid; bicyclo-prostaglandin E2; N6-acetyl-L-lysine.

The substrate may be any suitable surface which can carry an antibody. For example, the substrate may be plastic, for example a plate (e.g. a multi-well plate). Alternatively, the substrate may be a porous substrate. The porous substrate may be any material which allows another medium to pass through it. Any suitable porous substrate could be used, for example a woven material, or a cellulosic material.

The substrate carries the capture antibodies. The antibodies may be carried within the substrate or on the surface of the substrate. The antibodies may form a chemical interaction with the surface of the substrate. The antibodies may be bound to the substrate.

In embodiments, the device is a lateral flow device. Alternatively, the device may be a flow through device or an ELISA device.

Advantageously the device is a rapid, simple, non-invasive diagnostic providing quick diagnosis of paratuberculosis. It is easily accessible to farmers, for example, to improve detection and control of paratuberculosis. The device allows farmers to monitor paratuberculosis and specifically target treatment to those animals with paratuberculosis. A reliable on-farm diagnostic kit will help prevent disease, particularly the spread of disease, and increase the efficiency of livestock production.

Advantageously the device is a rapid, simple, non-invasive diagnostic providing quick diagnosis of Crohn's disease which can be used in locations remote from laboratories.

Lateral flow and flow through devices are particularly advantageous in that they can be used remotely to obtain rapid results in a simple manner.

A sample to be tested (i.e. a sample from the subject) may be applied to the immunological capture device, for example to the substrate of the immunological capture device.

A second antibody may also be applied the substrate of the immunological capture device. The second antibody may be specific to the metabolites or to the capture antibodies (i.e. the antibodies carried on the substrate). The second antibody may have coloured particles attached. The coloured particles may be covalently linked to the second antibody. The coloured particles may be cellulose beads or plastic microparticles, for example. In alternative embodiments the second antibody may be linked to an enzyme, via bio-conjugation, for example. In such embodiments, a composition comprising a substrate which undergoes a colour change upon reaction with the enzyme, indicating the presence of the enzyme, may be added to the device during use. Suitable enzymes and compositions will be well known to the skilled person.

In embodiments in which the second antibody is specific to the metabolites, binding of the second antibody to the metabolites bound to the capture antibodies results in a colour change. The presence of the metabolites can therefore be detected by a colour change.

In embodiments in which the second antibody is specific to the capture antibodies, binding of the second antibody to the capture antibody may take place when the metabolite is not present. In such an embodiment, binding of the second antibody to the capture antibodies, results in a colour change. The absence of the metabolites can therefore be detected by a colour change.

A reader, for example a lateral flow reader may be used to quantify the colour intensity.

In embodiments in which the device is a lateral flow or flow through device, the device may further comprise a control line. Colouring of the control line indicates successful completion of the test.

In embodiments in which the device is a lateral flow or flow through device, the device may further comprise a test line. The test line may comprise capture antibodies to the one or more metabolites.

Colouring of the test line indicates the presence or absence of the metabolites in the sample (as discussed above). Comparison of the coloured intensity of the control line and test line can be used to indicate the metabolite level in the sample and therefore whether the sample is from a subject in which Mycobacterium avium paratuberculosis, paratuberculosis or Crohn's disease is present. A reader, for example a lateral flow reader may be used to quantify the coloured intensity of the control and test lines.

In embodiments, the device may further comprise a housing. The substrate may be positioned within the housing.

The device can be used to detect metabolites in blood serum and whole blood samples, for example.

Preferably the device provides a test result in 60 minutes or less from test initiation. The device may provide a test result in 30 minutes or less, 20 minutes or less or 10 minutes or less from test initiation.

The device may be used in the method of the invention.

The present invention also provides a kit for determining the presence of Mycobacterium avium paratuberculosis, paratuberculosis or Crohn's disease in a subject, the kit comprising:
(i) an immunological capture device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol; and
(ii) a second antibody, wherein the second antibody is specific to the one or more metabolites or to the capture antibodies.

The device and second antibody of the kit are described above in relation to the immunological capture device of the present invention. The kit may be used in the method of the invention.

The present invention also provides the use of one or more metabolites for detecting Mycobacterium avium paratuberculosis in a subject, the one or more metabolites being selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The present invention also provides the use of one or more metabolites for detecting paratuberculosis in a subject, the one or more metabolites being selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The present invention also provides the use of one or more metabolites for detecting Crohn's disease in a subject, the one or more metabolites being selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

The described and illustrated embodiments are to be considered as illustrative and not restrictive in character, it being understood that only the preferred embodiments have been shown and described and that all changes and modifications that come within the scope of the inventions as defined in the claims are desired to be protected.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

### DETAILED DESCRIPTION OF THE INVENTION

The present invention will now be further described with reference to the following figures which show:

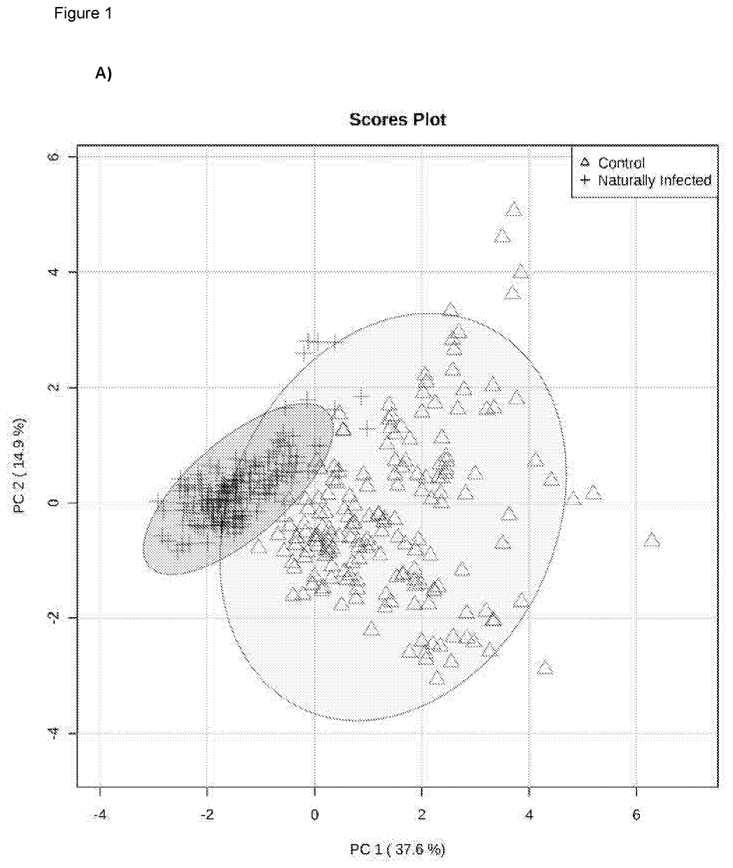

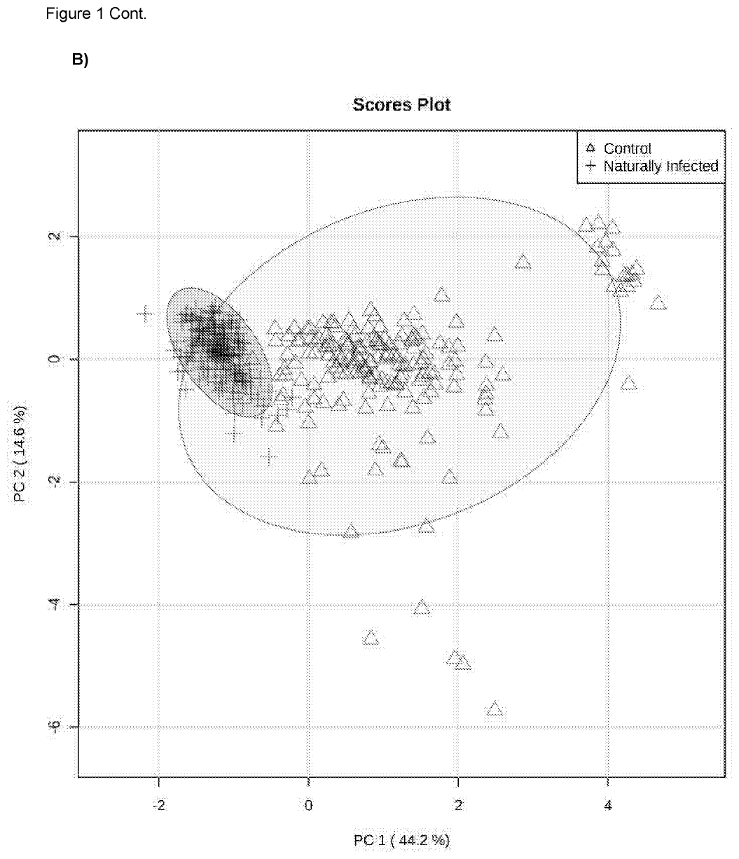

FIG. 1: Principal component analyses for naturally MAP infected and control heifers in the (A) negative ionization and (B) positive ionization modes. The larger ellipses represent 95% confidence intervals for each experimental class.

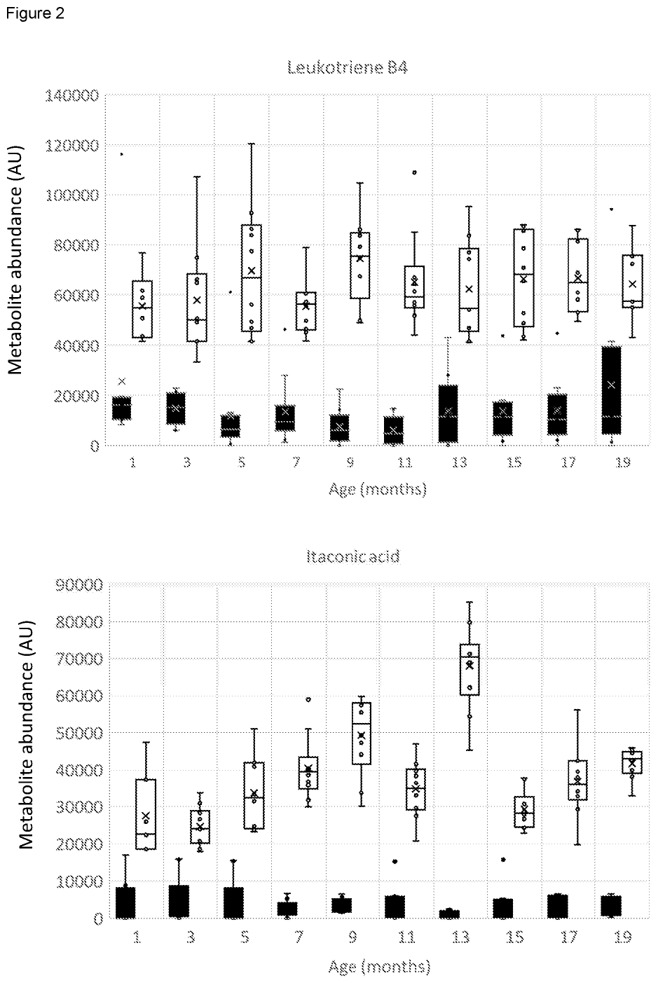

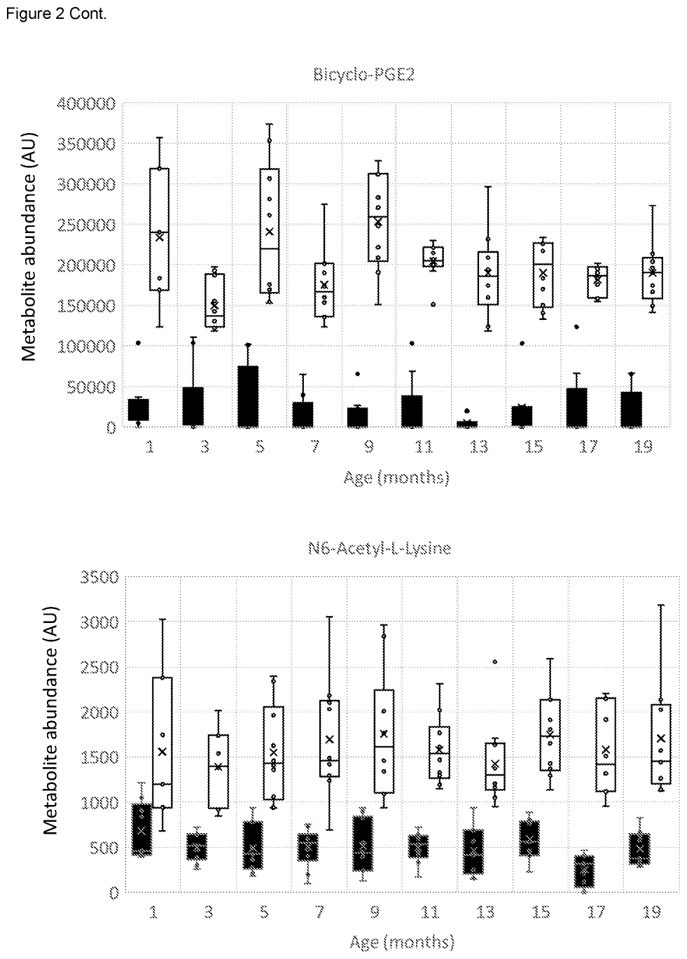

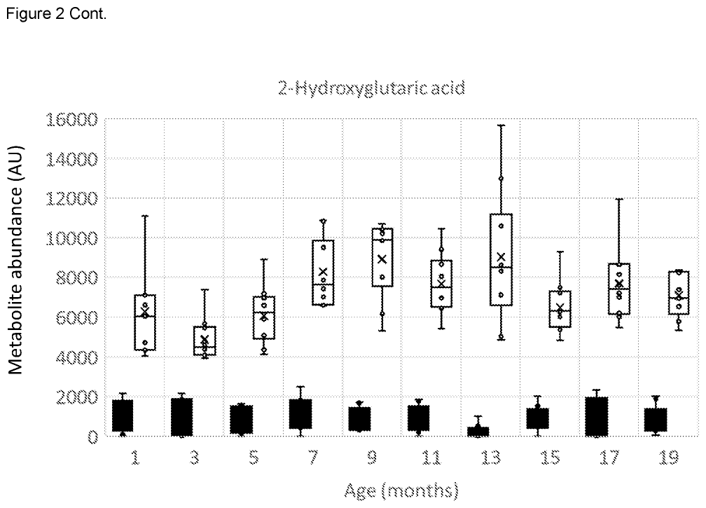

FIG. 2: Box and whisker plots of metabolites which display minimal overlapping between groups, naturally MAP infected and control heifers, aged ˜1-month to 19-months. Grey boxplots=naturally MAP infected heifers, Black boxplots=control heifers.

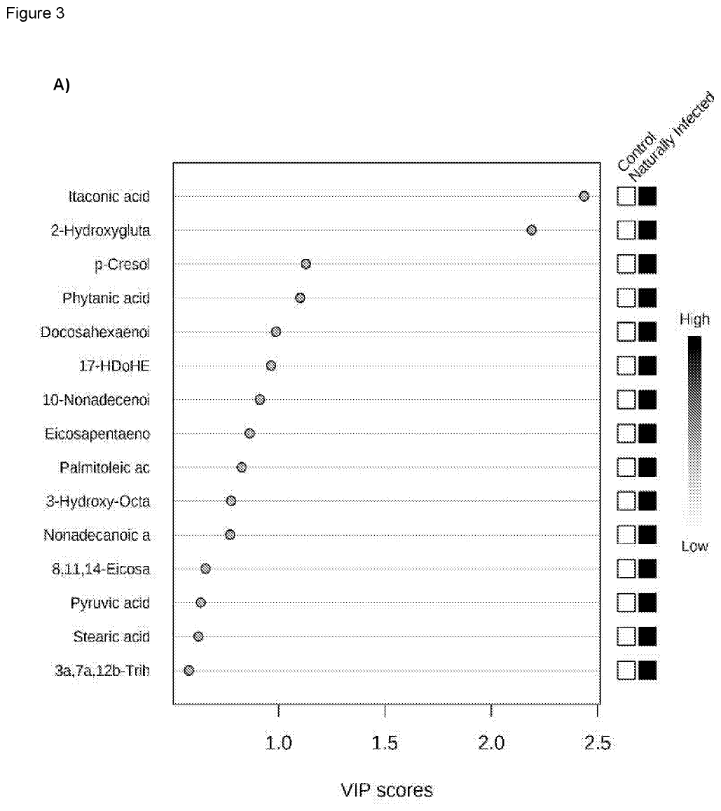

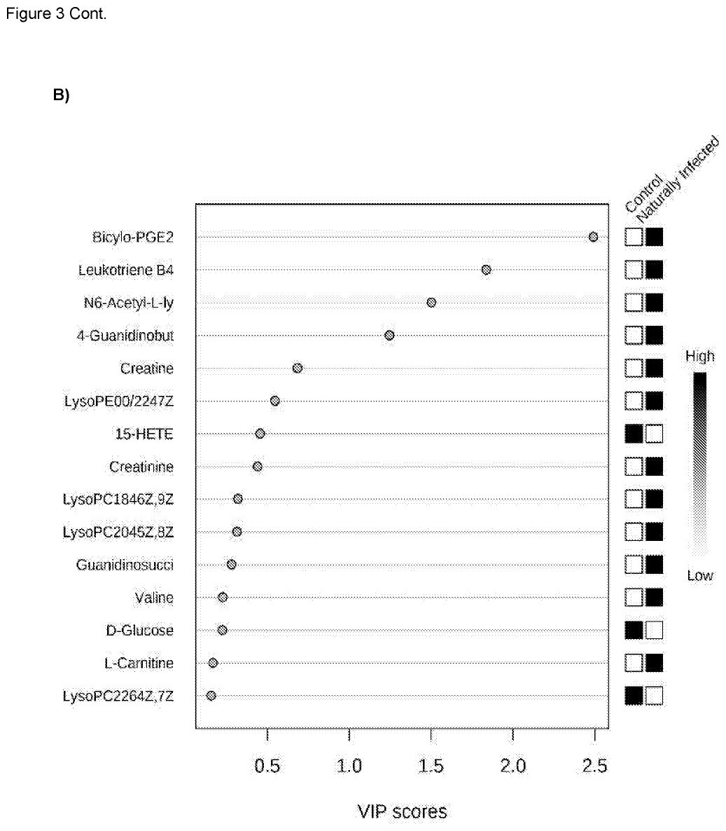

FIG. 3: VIP score plots produced by partial least squares—discriminate analysis (PLS-DA) of metabolites differentially expressed in naturally MAP infected and control heifers in the (A) negative ionization mode m/z and (B) positive ionization mode m/z.

As discussed above, the method of the present invention allows the presence of Mycobacterium avium paratuberculosis in a subject to be detected. The method of the present invention therefore allows the presence of paratuberculosis, for example subclinical paratuberculosis, to be detected. The present inventors undertook significant investigation to develop the method of the present invention and identified a number of metabolites, the level of which is significantly altered in subjects in which

Mycobacterium avium paratuberculosis is present. The present inventors have therefore identified a subset of biomarkers which can be used to quickly and accurately identify animals affected by paratuberculosis, so that they can be treated accordingly. The biomarker subset can also be used to identify subjects affected by Crohn's disease, a disease for which Mycobacterium avium paratuberculosis has long been considered a causative agent.

Materials and Methods

Animal Samples

Samples can be obtained from cattle using a 5 ml vacutainer of different types (classified based on tap colours to isolate either blood serum (yellow top) or plasma (purple top)).

The data described herein were generated from biobanked sera from 20 heifers (10 naturally MAP infected and 10 age-matched control heifers), aged between 1-month and 19-months were utilised. Female, Holstein-Friesian heifer calves were sourced from MAP-free or low prevalence herds and housed at the specific pathogen free (SPF) facilities at the Institute for Animal Disease Control (CIDC) in Lelystad, the Netherlands. Animals were housed according to their MAP status, which was determined using faecal culture. Naturally MAP infected and control heifers continued to undergo MAP faecal culture tests until death and 22-months of age, respectively. Experimental procedures were approved by the Ethical Committee of the CIDC.

Untargeted metabolite fingerprinting by flow infusion electrospray ionization high resolution mass spectrometry (FIE-HRMS)

The Figures presented herein were generated based on data obtained by FIE-HRMS. In flow infusion, samples are injected directly into a solvent (usually methanol-water) line leading to a mass spectrometer. There is no pre-separation (as is the case with GC-MS or LC-MS).

To generate the data presented in the Figures, biobanked sera were defrosted on ice, vortexed for 5 seconds and 200 μl was pipetted into 1520 μl pre-chilled solvent mix (methanol/chloroform [4/1]) containing 1 micro-spoon of glass beads. Samples were then vortexed for 5 seconds, shaken for 15 minutes at 4° C. and kept at −20° C. for 20 minutes. Following centrifugation at 14000 rpm at 4° C. for 5 minutes, 100 μl of the plasma supernatant was transferred into mass spectrometry vials along with 100 μl methanol/water [70/30]. Samples were stored at −80° C. until analysis using FIE-HRMS. For each sample, 20 μl were injected into a flow of 60 μl per minute water-methanol, at a ratio of 70% water and 30% methanol, using a Surveyor flow system into a Q Exactive plus mass analyser instrument (Thermo Fisher Scientific©, Bremen, Germany) for high throughput FIE-HRMS. Data acquisition for each serum sample was done by alternating the positive and negative ionisation modes, throughout four different scan ranges (15-110 m/z, 100-220 m/z, 210-510 m/z, 500-1200 m/z) with an acquisition time of 2 minutes.

Statistical Analysis

To generate the Figures presented herein, metabolomic data were analysed using R language-based programmes (Chong et al., 2019). Data were subjected to interquartile range-based filtering, logo transformations and pareto scaling. Univariate analyses used false discovery rate (FDR) adjusted t-tests to identify m/z which significantly (p-values<0.05) differed between experimental classes. Variables of importance for the projection (VIP) scores (>2) following multivariate analyses were also used to indicated m/z which discriminated between the classes. Repeated measures t-tests were used to assess m/z that differentially accumulated in naturally MAP infected heifers over time using SPSS version 24 (IBM SPSS Statistics for Windows., New York, USA). Principal component analysis (PCA) and partial least squares-discriminate analysis (PLS-DA) visualised the differences between the experimental classes. Major sources of variation were displayed using unsupervised hierarchical clustering analysis (HCA) and VIP score plots. Area under the curve (AUC) based on sensitivity and specificity estimates was used use determine the accuracy of the target m/z as potential biomarkers. Random Forest (RF) was used as an alternative multivariate classification test. This uses a confusion matrix to estimate how often given m/z variable would give an estimate of the classification error.

Metabolites were identified using the DIMEdb database based on their m/z, molecular formula and the Bovine Metabolome Database (Foroutan et al., 2020). All isotopes/adducts were considered.

Results

MAP Status

FIE-HRMS assessments were based on biobanked sera from naturally MAP infected and age-matched control heifers, aged between 1-month and 19-months. The MAP status of heifers was determined using faecal culture. Naturally MAP infected heifers were tested monthly using LJ growth media and IS900 PCR. MAP faecal shedding was observed in 9 out of 10 heifers and in the remaining heifer at 40-months of age. Control heifers were tested periodically using LJ growth media and remained MAP negative.

Metabolomic Changes

PCA of the serum metabolite profiles derived from both negative (FIG. 1A) and positive ionisation modes m/z (FIG. 1B) discriminated between naturally MAP infected and control heifers with minimal overlap between the 95% confidence intervals for each class. Control heifers showed greater variation compared to naturally MAP infected heifers.

Analyses identified 38 metabolites which differentially accumulated in naturally MAP infected heifers (Table 1). The MAP-independent effects of dietary changes, growth and development were assessed using repeated measures ANOVA and visualized using estimated marginal means plots. Analyses indicated 30/38 metabolites and 26/38 metabolites being significantly affected by time and MAP status*time respectively. Estimated marginal means plots showed that the accumulation of some metabolites was consistently higher in naturally MAP infected heifers (see FIG. 2, for example), many of these metabolites displaying individual diagnostic accuracies (area under the curve; AUC) of over 70% (the minimum value that can be considered useful as biomarker) (Table 2). Thus, these metabolites could make particularly effective biomarkers in differentiating between naturally MAP infected and control heifers at all stages of growth and development.

Alongside t tests, VIP score plots produced by PLS-DA highlighted the most important metabolites when discriminating between naturally MAP infected and control heifers. Interestingly, the expression of the 5 metabolites with the highest VIP scores were consistently higher in naturally MAP infected heifers than control heifers, as shown by estimated marginal means plots (FIG. 2). These metabolites included itaconic acid and 2-hydroxyglutaric acid in negative ionization, (FIG. 3A) and bicyclo-prostaglandin E2, leukotriene B4, N6-acetyl-L-lysine and 4-guanidinobutanoic acid, in positive ionization (FIG. 3B). These metabolites demonstrated area under the curve (AUC) values of >0.95, range 0.966-1.000 (Table 2). When random forest assessments were used very low classification class errors between naturally MAP infected and control heifers of 0.000 and 0.00552 were derived.

Taken together, the five metabolites showing highest levels of consistent differentiation between naturally MAP infected and control heifers (leukotriene B4, itaconic acid, bicyclo-prostaglandin E2, N6-acetyl-L-lysine, 2-hydroxyglutaric acid) represent a metabolite signature which targets pre-symptomatic MAP infected cattle with a pooled accuracy of >95% (Table 3).

To better understand the biological relevance of metabolomic changes within naturally MAP infected heifers, these metabolites were located on KEGG global metabolic network maps. These indicated the prominence of fatty acyls in the negatively ionised metabolites and arachidonic acid derivatives in the positively ionised metabolites. However, when these metabolites were assessed via MSEA using ORA, only the alpha linoleic acid and linoleic acid metabolism was significantly enhanced. Nevertheless, 19 of the identified metabolites belonged to the fatty acyl class and of these, 16 to the fatty acids and conjugates sub-class. These changes suggest that immune and inflammatory events are key as MAP colonization progressed.

TABLE 1
Metabolite levels within naturally MAP infected and control heifers.

| | | | | P-values | | |
|---|---|---|---|---|---|---|
| | | | | | | MAP |
| | MAP Status | | | MAP | | Status × |
| Common Metabolite Names | Infected | Control | SEM | Status | Time | Time |
| 16-Methylheptadecanoic acid | 880.097 | 428.605 | 101.589 | 8.66E−18 | 0.000 | 0.006 |
| 17-HDoHE | 2092.433 | 956.798 | 232.590 | 7.38E−20 | 0.077 | 0.099 |
| 2-Hydroxyglutaric acid | 6968.017 | 829.932 | 418.733 | 6.20E−59 | 0.000 | 0.000 |
| 3-Hydroxy-Octadecanoic acid | 2277.269 | 1119.311 | 182.751 | 3.10E−29 | 0.000 | 0.002 |
| 3a,7a,12b-Trihydroxy-5b-cholanoic | 27463.857 | 13886.675 | 6,761.888 | 1.25E−04 | 0.010 | 0.060 |
| acid | | | | | | |
| 4-Guanidinobutanoic acid | 959.263 | 357.268 | 84.874 | 1.22E−46 | 0.002 | 0.000 |
| 5-HEPE | 19796.357 | 24731.243 | 1,298.340 | 8.61E−21 | 0.123 | 0.114 |
| 8,11,14,17-Eicosatetraenoic acid | 24953.244 | 18131.100 | 2,201.512 | 4.62E−13 | 0.159 | 0.065 |
| 8,11,14-Eicosatrienoic acid | 13471.191 | 8574.041 | 915.540 | 4.19E−24 | 0.029 | 0.003 |
| 9,10-DHOME | 3745.790 | 3070.854 | 195.904 | 2.03E−10 | 0.061 | 0.009 |
| 10-Nonadecenoic acid | 2010.000 | 1044.322 | 191.240 | 1.18E−21 | 0.006 | 0.025 |
| Adrenic acid | 1546.148 | 1151.444 | 152.728 | 1.63E−06 | 0.026 | 0.021 |
| Arachidonic acid | 4002.095 | 3121.539 | 377.465 | 8.46E−13 | 0.004 | 0.005 |
| Bicyclo-PGE2 | 202290.422 | 21399.737 | 13,530.638 | 1.15E−80 | 0.010 | 0.007 |
| Carnitine | 4130.360 | 3979.879 | 429.034 | 1.21E−02 | 0.000 | 0.000 |
| Creatine | 18525.418 | 12709.354 | 978.194 | 1.02E−38 | 0.080 | 0.017 |
| Creatinine | 64984.861 | 53091.855 | 2,235.817 | 4.35E−27 | 0.407 | 0.029 |
| Glucose | 283122.175 | 357303.877 | 31,674.354 | 1.57E−02 | 0.002 | 0.013 |
| DHAP(18:0) | 1928.880 | 1868.962 | 228.679 | 2.49E−03 | 0.004 | 0.000 |
| Docosahexaenoic acid | 3556.592 | 1648.910 | 393.141 | 1.97E−22 | 0.007 | 0.034 |
| Eicosapentaenoic acid | 11641.734 | 6102.013 | 969.370 | 4.60E−19 | 0.000 | 0.051 |
| Guanidinosuccinic acid | 23675.293 | 21598.587 | 1,165.107 | 1.44E−07 | 0.002 | 0.015 |
| Itaconic acid | 38318.702 | 3484.773 | 1,874.525 | 9.96E−68 | 0.000 | 0.000 |
| Leukotriene B4 | 67160.959 | 14524.999 | 7,361.879 | 4.61E−29 | 0.746 | 0.197 |
| LPA(18:2(9Z,12Z)/0:0) | 1982.385 | 3130.118 | 423.917 | 1.10E−08 | 0.009 | 0.347 |
| LysoPC(18:4(6Z,9Z,12Z,15Z)) | 51011.667 | 42324.199 | 3,459.485 | 3.73E−08 | 0.160 | 0.200 |
| LysoPC(20:4(5Z,8Z,11Z,14Z)) | 692622.065 | 590103.540 | 43,247.009 | 1.33E−08 | 0.027 | 0.025 |
| LysoPC(22:6(4Z,7Z,10Z,13Z,16Z,19Z)) | 53983.004 | 56544.212 | 5,268.892 | 2.34E−02 | 0.016 | 0.076 |
| LysoPE(0:0/22:4(7Z,10Z,13Z,16Z)) | 38288.811 | 27641.890 | 3,715.051 | 1.58E−06 | 0.001 | 0.131 |
| N6-Acetyl-L-lysine | 1691.204 | 498.729 | 147.912 | 1.04E−60 | 0.090 | 0.117 |
| Nonadecanoic acid | 1501.629 | 860.373 | 125.330 | 2.63E−20 | 0.000 | 0.086 |
| Palmitic acid | 225984.794 | 205205.885 | 12,735.750 | 4.67E−10 | 0.009 | 0.053 |
| Palmitoleic acid | 7226.218 | 4337.230 | 742.694 | 1.62E−30 | 0.001 | 0.007 |
| p-Cresol | 15785.626 | 11468.347 | 2,543.193 | 3.17E−14 | 0.000 | 0.008 |
| Phytanic acid | 13273.899 | 5807.038 | 1,226.896 | 9.27E−20 | 0.000 | 0.004 |
| Pyruvic acid | 25919.185 | 18928.077 | 2,437.983 | 1.50E−15 | 0.003 | 0.004 |
| Stearic acid | 212529.882 | 145180.423 | 16,168.671 | 1.56E−24 | 0.004 | 0.056 |
| Valine | 6993.263 | 6390.668 | 764.050 | 5.63E−03 | 0.011 | 0.007 |

TABLE 2
Metabolites differentially expressed in naturally MAP infected and control heifers

| | | Common Metabolite | | | | |
|---|---|---|---|---|---|---|
| Class | Sub Class | Name | Mode | AUC | P-value | Log2(FC) |
| Carboxylic | Amino acids, | N6-Acetyl-L-lysine | Pos | 0.995 | 1.04E−60 | −2.0685 |
| acids and | peptides, and | 4-Guanidinobutanoic | Pos | 0.9232 | 1.22E−46 | −1.5717 |
| derivatives | analogues | acid | | | | |
| | | Creatine | Pos | 0.886 | 1.02E−38 | −0.50871 |
| | | Creatinine | Pos | 0.8221 | 4.35E−27 | −0.2471 |
| | | Guanidinosuccinic acid | Pos | 0.6439 | 1.44E−07 | −0.19556 |
| | | Valine | Pos | 0.5874 | 0.005629 | −0.23982 |
| Fatty Acyls | Eicosanoids | Bicyclo-PGE2 | Pos | 0.9994 | 1.15E−80 | −5.1846 |
| | | Leukotriene B4 | Pos | 0.9655 | 4.61E−29 | −4.1232 |
| | | 5-HEPE | Pos | 0.7769 | 8.61E−21 | 0.29914 |
| | Fatty acids and | Itaconic acid | Neg | 0.9999 | 9.96E−68 | −5.392 |
| | conjugates | Docosahexaenoic acid | Neg | 0.8676 | 1.97E−22 | −1.3874 |
| | | cis-10-Nonadecenoic | Neg | 0.8533 | 1.18E−21 | −1.2039 |
| | | acid | | | | |
| | | 17-HDoHE | Neg | 0.8495 | 7.38E−20 | −1.4021 |
| | | Palmitoleic acid | Neg | 0.849 | 1.62E−30 | −0.84642 |
| | | R-3-Hydroxy-Octadecanoic | Neg | 0.8236 | 3.10E−29 | −0.76056 |
| | | acid | | | | |
| | | 8,11,14-Eicosatrienoic | Neg | 0.8189 | 4.19E−24 | −0.59477 |
| | | acid | | | | |
| | | Nonadecanoic acid | Neg | 0.8044 | 2.63E−20 | −0.89077 |
| | | Stearic acid | Neg | 0.7975 | 1.56E−24 | −0.53114 |
| | | Eicosapentaenoic acid | Neg | 0.7912 | 4.60E−19 | −1.1494 |
| | | 16-Methylheptadecanoic | Neg | 0.7898 | 8.66E−18 | −0.97126 |
| | | acid | | | | |
| | | Arachidonic acid | Neg | 0.7353 | 8.46E−13 | −0.38789 |
| | | 9,10-DHOME | Neg | 0.7241 | 2.03E−10 | −0.24916 |
| | | 8,11,14,17-Eicosatetraenoic | Neg | 0.7241 | 4.62E−13 | −0.41624 |
| | | acid | | | | |
| | | Palmitic acid | Neg | 0.7048 | 4.67E−10 | −0.22981 |
| | | Adrenic acid | Neg | 0.644 | 1.63E−06 | −0.36134 |
| Glycero- | Glycero- | LPA(18:2(9Z,12Z)/0:0) | Neg | 0.6774 | 1.10E−08 | 0.53477 |
| phospholipids | phosphates | | | | | |
| | Glycero- | LysoPC(20:4(5Z,8Z,11Z,14Z)) | Pos | 0.6722 | 1.33E−08 | −0.2265 |
| | phosphocholines | LysoPC(18:4(6Z,9Z,12Z,15Z)) | Pos | 0.661 | 3.73E−08 | −0.24245 |
| | | LysoPC(22:6(4Z,7Z,10Z,13Z,16Z,19Z)) | Pos | 0.5945 | 0.023415 | 0.13975 |
| | Glycero- | LysoPE00/2247Z,10Z,13Z,16Z | Pos | 0.6614 | 1.58E−06 | −0.80084 |
| | phosphoethanolamines | | | | | |
| Hydroxy | Short chain hydroxy | 2-Hydroxyglutaric acid | Neg | 0.9998 | 6.20E−59 | −4.5575 |
| acids and | acids and derivatives | | | | | |
| derivatives | | | | | | |
| Keto acids | Alpha-keto acids and | Pyruvic acid | Neg | 0.7465 | 1.50E−15 | −0.68579 |
| and derivatives | derivatives | | | | | |
| Organooxygen | Carbohydrates and | D-Glucose | Pos | 0.591 | 0.015674 | 0.27051 |
| compounds | carbohydrate | | | | | |
| | conjugates | | | | | |
| | Carbonyl compounds | DHAP(18:0) | Neg | 0.635 | 0.002491 | −0.20646 |
| Phenols | Cresols | p-Cresol | Neg | 0.7364 | 3.17E−14 | −2.2681 |
| Prenol lipids | Diterpenoids | Phytanic acid | Neg | 0.8226 | 9.27E−20 | −1.8344 |
| Quaternary | Carnitines | L-Carnitine | Pos | 0.6066 | 0.012137 | −0.14492 |
| ammonium | | | | | | |
| salts | | | | | | |
| Steroids and | Bile acids, alcohols | 3a,7a,12b-Trihydroxy-5b-cholanoic | Neg | 0.6444 | 1.25E−04 | −1.1495 |
| steroid | and derivatives | acid | | | | |
| derivatives | | | | | | |

1 = Ionisation mode m/z
2 = Area under the curve
3 = P-value comparing MAP status effect results from t-test using an adjusted P-value (FDR) cut-off of 0.05
4 = Log2(FC) (fold change) in naturally MAP infected heifers

TABLE 3
Multivariate AUC (area under the ROC curve) of leukotriene
B4, itaconic acid, bicyclo-prostaglandin E2, N6-acetyl-
L-lysine and 2-hydroxyglutaric acid.

| Number of | | |
|---|---|---|
| biomarkers | AUC | 95% CI |
| 2 | 1 | 0.999-1 |
| 3 | 1 | 0.999-1 |
| 4 | 1 | 0.9999-1 |
| 5 | 1 | 1-1 |

Discussion

Few studies have used omic techniques to study MAP, particularly metabolomics, despite their ability to demonstrate the dynamics of infection and highlight potential diagnostic biomarkers. The present inventors have undertaken significant testing to demonstrate the utility of metabolomics in the study of MAP and the derivation of metabolite biomarkers. The inventors metabolomic approach based on FIE-HRMS followed by the metabolite identification proved to be an effective means to provide important insights to MAP progression. Based on the identified metabolites key biochemical pathways, chemical taxonomy classes and subclasses were targeted. In total, 38 metabolites could discriminate between MAP-infected, compared to control heifers (Tables 1 and 2), 6 of which exhibited a significant correlation to established MAP antigens (FIG. 3).

Five metabolites (leukotriene B4, itaconic acid, bicyclo-prostaglandin E2, N6-acetyl-L-lysine, 2-hydroxyglutaric acid) were found to be significantly elevated in naturally MAP infected heifers throughout the trial and therefore represent particularly effective biomarkers for use in identifying subclinical MAP infections, at a time when clinical symptoms are not apparent.

Metabolites within the amino acids, peptides, and analogues subclass (class—carboxylic acids and derivatives), 4-guanidinobutanoic acid, creatine, creatinine, guanidinosuccinic acid and N6-acetyl-L-lysine levels increased within MAP infected heifers. N6-acetyl-L-lysine levels were consistently higher within MAP infected heifers throughout the study and had an AUC accuracy of 0.995. N6-acetyl-L-lysine is a post translational modified version of lysine and its increases could reflect elevated lysine levels within the serum of MAP inoculated cattle as a result of a reduced ability of the gastrointestinal tract to absorb amino acids due to inflammation. Alternatively, MAP may be inducing elevated N6-acetyl-L-lysine expression within cattle to suppress the innate immune system.

Bicyclo-prostaglandin E2 (PGE2) was significantly elevated within naturally MAP infected heifers throughout the study and demonstrated an AUC accuracy of 0.999. Prostaglandins are synthesized from arachidonic acid to prostaglandin (PG) G2 and prostaglandin endoperoxide (PGH) by prostaglandin G/H synthase, (also known as cyclooxygenase, [COX]), before being converted into a range of eicosanoids including PGE2, PGD2 and PGF2a. PGE2 aids the regulation of cell proliferation, angiogenesis and inflammation.

Similar to PGE2, leukotriene B4 (LTB4) was significantly elevated within naturally MAP infected heifers with an AUC accuracy of 0.966 suggesting it as a potential biomarker. As with prostaglandins, leukotrienes are synthesised from arachidonic acid. In this case, arachidonic acid is converted to 5-hydroperoxyeicosatetraenoic acid and then to leukotriene A4 (LTA4) by 5-lipoxygenase (ALOXS). LTA4 is rapidly hydrolysed by LTA4 hydrolase, producing LTB4.

Primary metabolism is common to all organisms and changes in the serum metabolome suggested that this was being shifted in either the bacterium or host in MAP infected cattle. Changes in icatonic acid and 2-hydroxyglutaratic acid within naturally MAP infected heifers suggest that MAP acquires energy through the β-oxidation of fatty acids and glyoxylate shunt. The β-oxidation pathway converts fatty acids into acetyl CoA which can then be incorporated into the glyoxylate shunt or tricarboxylic acid (TCA) cycle. The glyoxylate shunt is a two-step metabolic pathway that bypasses the CO₂-producing steps of the TCA cycle and allows bacteria to access acetyl moieties from acetate, fatty acids, or ketogenic amino acids, as opposed to glucose.

Icatonic acid was significantly elevated within naturally MAP infected heifers throughout the study and produced an AUC accuracy of 0.999, so is another highly effective biomarker. Icatonic acid is produced from the decarboxylation of cis-aconitic acid within the TCA cycle by the immune-responsive gene 1 protein and demonstrates various roles including inhibiting the glyoxylate shunt, inhibiting bacterial growth and promoting macrophage recruitment.

Likewise, 2-hydroxyglutaratic acid was also significantly elevated within naturally MAP infected heifers throughout the study and displayed a very similar AUC accuracy of 0.999. 2-hydroxyglutarate is produced from α-ketoglutarate using isocitrate dehydrogenase 1 or 3-phosphoglycerate dehydrogenase and isocitrate dehydrogenase is located at the point where the TCA cycle and glyoxylate shunt differentiate. Unlike other intracellular bacteria which lack a-ketoglutarate dehydrogenase MAP utilises 2-oxoglutarate, enabling TCA cycle progression.

Additionally, functions of 2-hydroxyglutaric acid include cell respiration, regulation of the NF-κB pathway and CD8+ T-cell differentiation. It is possible therefore that the host could exploit MAP promoting 2-hydroxyglutarate production to aid the immune system response through promoting CD8+ T-cell differentiation. These results suggest that MAP can acquire energy from the TCA cycle or glyoxylate shunt and provides further proof a sustained immune system response.

Conclusion

Metabolomic analysis of naturally MAP infected heifers and control heifers, demonstrated the ability of untargeted FIE-MS to examine the metabolic processes of mycobacterial infections. The inventors findings showed clear differentiation between naturally MAP infected and control heifers aged between 1-month and 19-months of age, including five (leukotriene B4, itaconic acid, bicyclo-prostaglandin E2, N6-acetyl-L-lysine, 2-hydroxyglutaric acid) which were significantly elevated in naturally MAP infected heifers throughout the trial.

Taken together, the five metabolites showing highest levels of consistent differentiation between naturally MAP infected and control heifers (leukotriene B4, itaconic acid, bicyclo-prostaglandin E2, N6-acetyl-L-lysine, 2-hydroxyglutaric acid) represent a metabolite signature which targets pre-symptomatic MAP infected cattle with a pooled accuracy of >95%. These metabolites demonstrate greater sensitivity and specificity when predicting MAP infection than the commercial diagnostic tests currently available and are therefore extremely effective biomarkers for use in identifying subclinical MAP infections. By using the five metabolite levels in combination, the present inventors have developed a highly effective diagnostic fingerprint for identifying MAP infection.

It will be appreciated that numerous modifications to the above-described method may be made without departing from the scope of the invention as defined in the appended claims. For example, although the five metabolites showing highest levels of consistent differentiation between naturally MAP infected and control heifers (leukotriene B4, itaconic acid, bicyclo-prostaglandin E2, N6-acetyl-L-lysine, 2-hydroxyglutaric acid) are described as being used together as a diagnostic fingerprint, the skilled person will appreciate that other metabolite combinations are envisaged and would provide effective biomarkers for MAP infection.

### REFERENCES

Barratt, A. S., Arnoult, M. H., Ahmadi, B. V., Rich, K. M., Gunn, G. J. and Stott, A. W. (2018). A framework for estimating society's economic welfare following the introduction of an animal disease: The case of Johne's disease. PLoS ONE. 13:6. doi: e0198436.

Beckmann, M., Parker, D., Enot, D. P., Duval, E. and Draper, J. (2008). High-throughput, nontargeted metabolite fingerprinting using nominal mass flow injection electrospray mass spectrometry. Nature Protocols. 3:3. doi: 10.1038/nprot.2007.500.

Chong, J., Wishart, D. and Xai, J. (2019). Using MetaboAnalyst 4.0 for comprehensive and integrative metabolomics data analysis. Current Protocols in Bioinformatics. 68:1. doi: 10.1002/cpbi.86.

DEFRA et al., 2009 Cook, A. J. C., McGoldrick, A., Vidal-Diez, A. and Sayers, R. (2008). Chapter 3—Results of culture of pooled faecal samples from the participating herds. Pages 39-50 in SB4022: An integrated strategy to determine the herd level prevalence of Johne's disease in the UK dairy herd. http://archive.defra.gov.uk/foodfarm/farmanimal/diseases/atoz/documents/johnes-report0911.pdf. [Accessed Sep. 2, 2020].

Foroutan, A., Fitzsimmons, C., Mandal, R., Piri-Moghadam, H., Zheng, J., Guo, A. C. et al., (2020). The bovine metabolome. Metabolites. 20:233. doi: 10.3390/metabo10060233.

Naser S A, Ghobrial G, Romero C, Valentine J F. Culture of Mycobacterium avium subspecies paratuberculosis from the blood of patients with Crohn's disease. Lancet. 2004 Sep. 18-24;364(9439):1039-44. doi: 10.1016/S0140-6736(04)17058-X. PMID: 15380962.

The UniProt Consortium. (2018). UniProt: a worldwide hub of protein knowledge. Nucleic Acids Research. 47:D1. doi: 10.1093/nar/gky1049.

1. A method for determining the presence of Mycobacterium avium paratuberculosis in a subject, the method comprising the steps of:
(i) determining the level of one or more metabolites in a sample from the subject;
(ii) comparing the level of said one or more metabolites with the level of said one or more metabolites in a control sample to determine whether Mycobacterium avium paratuberculosis is present in the subject;
wherein the one or more metabolites are selected from: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

2. The method according to claim 1, wherein the one or more metabolites are selected from 2-hydroxyglutaric acid; leukotriene B4; itaconic acid; bicyclo-prostaglandin E2; N6-acetyl-L-lysine; guanidinobutanoic acid; creatine;
docosahexaenoic acid; and cis-10-nonadecenoic acid

3. The method according to claim 1, wherein the one or more metabolites are selected from 2-hydroxyglutaric acid; leukotriene B4; itaconic acid; bicyclo-prostaglandin E2; and N6-acetyl-L-lysine.

4. The method according to claim 1, wherein the method comprises determining the level of five metabolites in the sample from the subject.

5. The method according to claim 1 wherein when the one or more metabolites are selected from: 16-methylheptadecanoic acid; 17-hydroxy docosahexaenoic acid; 2-hydroxyglutaric acid; R-3-hydroxy-octadecanoic acid; guanidinobutanoic acid; 8,11,14,17-eicosatetraenoic acid; 8,11,14-eicosatrienoic acid; 9,10-dihydroxy-12-octadecenoic acid; nonadecanoic acid; arachidonic acid; bicyclo-prostaglandin E2; creatinine; creatine; docosahexaenoic acid; eicosapentaenoic acid; itaconic acid; N6-acetyl-L-lysine; leukotriene B4; palmitic acid; palmitoleic acid; p-cresol; phytanic acid; pyruvic acid; stearic acid; and cis-10-nonadecenoic acid, a metabolite level in the sample which is higher than the metabolite level in the control sample indicates that Mycobacterium avium paratuberculosis is present in the subject.

6. The method according to claim 1, wherein the method comprises determining the level of 2-hydroxyglutaric acid; leukotriene B4;
itaconic acid; bicyclo-prostaglandin E2; and N6-acetyl-L-lysine in a sample, and wherein a metabolite level in the sample from the subject which is higher than the metabolite level in the control sample indicates that Mycobacterium avium paratuberculosis is present in the subject.

7. The method according to claim 1, wherein the subject is a ruminant.

8. The method according to claim 1, wherein the method comprises a method for determining the presence of paratuberculosis in a subject.

9. The method according to claim 1, wherein the method comprises a method for determining the presence of Crohn's disease in a subject.

10. An immunological capture device for detecting Mycobacterium avium paratuberculosis in a subject, the device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol.

11. The device according to claim 10, wherein the device is for detecting paratuberculosis or Crohn's disease in a subject.

12. The device according to claim 10 wherein the device comprises capture antibodies to two, three, four or five of the following metabolites: 2-hydroxyglutaric acid; leukotriene B4; itaconic acid; bicyclo-prostaglandin E2; N6-acetyl-L-lysine.

13. The device according to claim 10 wherein the device comprises capture antibodies to 2-hydroxyglutaric acid; leukotriene B4; itaconic acid; bicyclo-prostaglandin E2; and N6-acetyl-L-lysine.

14. A kit for determining the presence of Mycobacterium avium paratuberculosis in a subject, the kit comprising:
(i) an immunological capture device comprising a substrate carrying capture antibodies to one or more of the following metabolites: 2-hydroxyglutaric acid; leukotriene B4; itaconic acid; bicyclo-prostaglandin E2; N6-acetyl-L-lysine; guanidinobutanoic acid; creatine; docosahexaenoic acid; cis-10-nonadecenoic acid; creatinine; 5-hydroxyeicosapentaenoic acid, 17-hydroxy docosahexaenoic acid, palmitoleic acid; R-3-hydroxy-octadecanoic acid; 8,11,14-eicosatrienoic acid; nonadecanoic acid; stearic acid; eicosapentaenoic acid; 16-methylheptadecanoic acid; phytanic acid; arachidonic acid; 9,10-dihydroxy-12-octadecenoic acid, 8,11,14,17-eicosatetraenoic acid; palmitic acid; pyruvic acid; and p-cresol; and
(ii) a second antibody, wherein the second antibody is specific to the one or more metabolites or to the capture antibodies.

15. The kit according to claim 14 wherein the second antibody comprises a coloured particle covalently linked to the second antibody.
